(19) United States
(12) Patent Application Publication
(10) Pub. No.: US 2017/0007416 A1
(43) Pub. Date: Jan. 12, 2017

(54) HAMMER TOE IMPLANT

(71) Applicant: WRIGHT MEDICAL TECHNOLLOGY, INC., Memphis, TN (US)

(72) Inventors: Elizabeth J. SANDER, Memphis, TN (US); Christine M. PETTEYS, Bartlett, TN (US)

(21) Appl. No.: 14/419,194

(22) PCT Filed: Mar. 28, 2014
(86) PCT No.: PCT/US2014/032103

§ 371 Date: Feb. 2, 2015

(51) Int. Cl. A61F 2/42

(52) U.S. Cl. CPC A61F 2/4225; A61F 2002/30069

(57) ABSTRACT

An implant is disclosed including first and second blade portions comprising a plurality of serrated edges and an engagement portion connecting first and second blade portions, wherein the first and second blade portions are aligned along a common axis extending away from the engagement portion and both the first and second blade portions have a taper terminating at a point. In some embodiments, engagement portion is formed from a flexible or semi-flexible material.

### FIELD OF DISCLOSURE

The disclosed system relates to implants. More specifically, the disclosed system and method relate to an implant for treating hammer toe.

### BACKGROUND

Hammer toe is a deformity of the toe that affects the alignment of the bones adjacent to the proximal interphalangeal (PIP) joint. Hammer toe can cause pain and can lead to difficulty in walking or wearing shoes. A hammer toe can often result in an open sore or wound on the foot. In some instances, surgery may be required to correct the deformity by fusing one or both of the PIP and distal interphalangeal (DIP) joints.

The most common corrective surgery includes the placement of a pin or rod in the distal, middle, and proximal phalanxes of the foot to fuse the PIP and DIP joints. The pin or rod is cut at the tip of the toe, externally of the body. A plastic or polymeric ball is placed over the exposed end of the rod, which remains in the foot of the patient until the PIP and/or DIP joints are fused in approximately 6 to 12 weeks. This conventional treatment has several drawbacks such as preventing the patient from wearing closed toe shoes while the rod or pin is in place, and the plastic or polymeric ball may snag a bed sheet or other object due to it extending from the tip of the toe resulting in substantial pain for the patient.

Another conventional implant includes a pair of threaded members that are disposed within adjacent bones of a patient's foot. The implants are then coupled to one another through male-female connection mechanism, which is difficult to install in situ and has a tendency to separate.

Yet another conventional implant has a body including an oval head and a pair of feet, which are initially compressed. The implant is formed from nitinol and is refrigerated until it is ready to be installed. The head and feet of the implant expand due to the rising temperature of the implant to provide an outward force on the surrounding bone when installed. However, the temperature sensitive material may result in the implant deploying or expanding prior to being installed, which requires a new implant to be used.

In each of these potential treatments for hammer toe, the implant has a rigid design which prevents flexing the affected toe. This rigid implant design does not permit flexing and thus reduces natural motion in the joint of the affected toe.

Accordingly, an improved implant for treating hammer toe is desirable.

### SUMMARY

An improved implant for treating hammer toe is disclosed. An implant is disclosed including first and second blade portions comprising a plurality of serrated edges and an engagement portion connecting first and second blade portions, wherein the first and second blade portions are aligned along a common axis extending away from the engagement portion and both the first and second blade portions have a taper terminating at a point.

A method of treating hammer toe is disclosed. A method is disclosed comprising exposing a joint between first and second bones; resecting a respective end of the first and second bones; and installing an implant in the joint, where the implant comprises a first blade portion having a first taper terminating at a first point and a plurality of serrated edges configured to engage the first bone; a second blade portion having a second taper terminating at a second point and a plurality of serrated edges configured to engage the second bone; and an engagement portion connecting the first blade portion and the second blade portion, the engagement portion having a top spacer portion configured to be disposed between the first and second bones when the implant is installed.

In another embodiment, the present disclosure comprises a sleeve configured to be disposed about a circumference of the affected toe to improve tissue laxity and/or restrict joint movement.

In still a further embodiment, a spherical implant is configured to be disposed between two resected toe bones to space and enable natural anatomical movement of the resected toe bones.

### BRIEF DESCRIPTION OF THE DRAWINGS

These and other features and advantages of the present invention will be more fully disclosed in, or rendered obvious by the following detailed description of the preferred embodiments of the invention, which are to be considered together with the accompanying drawings wherein like numbers refer to like parts and further wherein:

FIG. 1A is a top profile view of one example of an improved implant for treating hammer toe in accordance with some embodiments of the present disclosure.

FIG. 1B is a side profile view of one example of an improved implant for treating hammer toe in accordance with some embodiments of the present disclosure.

FIG. 2A is a top profile view of one example of an improved implant for treating hammer toe in accordance with some embodiments of the present disclosure.

FIG. 2B is a side profile view of one example of an improved implant for treating hammer toe in accordance with some embodiments of the present disclosure.

FIG. 3A is a top profile view of one example of an improved implant for treating hammer toe in accordance with some embodiments of the present disclosure.

FIG. 3B is a side profile view of one example of an improved implant for treating hammer toe in accordance with some embodiments of the present disclosure.

FIG. 4A is a top profile view of one example of an improved implant for treating hammer toe in accordance with some embodiments of the present disclosure.

FIG. 4B is a side profile view of one example of an improved implant for treating hammer toe in accordance with some embodiments of the present disclosure.

FIG. 4C is a top profile view of an improved implant disposed between two resected toe bones in accordance with some embodiments of the present disclosure.

FIG. 5 is a top profile view of a sleeve disposed about a circumference of the affected toe in accordance with some embodiments of the present disclosure.

FIG. 6A is a top profile view of a spherical implant disposed between two resected toe bones in accordance with some embodiments of the present disclosure.

FIG. 6B is a side profile view of a spherical implant disposed between two resected toe bones in accordance with some embodiments of the present disclosure.

### DETAILED DESCRIPTION

This description of preferred embodiments is intended to be read in connection with the accompanying drawings, which are to be considered part of the entire written description. The drawing figures are not necessarily to scale and certain features of the invention may be shown exaggerated in scale or in somewhat schematic form in the interest of clarity and conciseness. In the description, relative terms such as “horizontal,” “vertical,” “up,” “down,” “top,” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral,” and the like are to be interpreted relative to one another or relative to an axis of elongation, or an axis or center of rotation, as appropriate. Terms concerning attachments, coupling, and the like, such as “connected” and “interconnected,” refer to a relationship wherein structures are secured or attached to one another either directly or indirectly through intervening structures, as well as both movable or rigid attachments or relationships, unless expressly described otherwise. The term “operatively connected” is such an attachment, coupling or connection that allows the pertinent structures to operate as intended by virtue of that relationship.

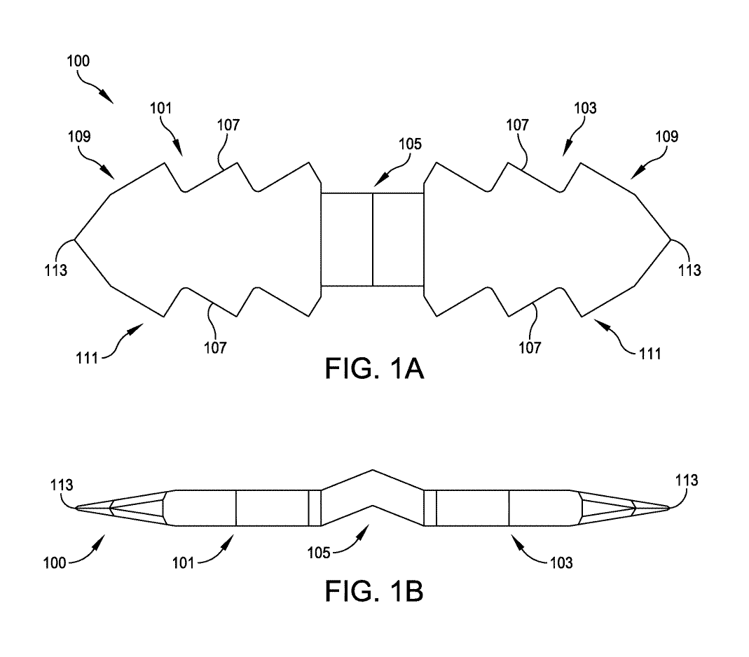

FIG. 1A is a top profile view of one example of an improved implant 100 for treating hammer toe in accordance with some embodiments of the present disclosure. FIG. 1B is a side profile view of one example of an improved implant 100 for treating hammer toe in accordance with some embodiments of the present disclosure. Implant 100 includes a first blade portion 101 and a second blade portion 103, which are connected together by an engagement portion 105. Implant 100 may have a substantially linear geometry.

First blade portion 101 and second blade portion 103 are configured to be inserted into bones in a toe and provide rotational and axial support for implant 100. Both first blade portion 101 and second blade portion 103 have a top edge 109 and bottom edge 111 which include a plurality of serrated edges 107. Serrated edges 107 help to maintain engagement between a toe bone and either first blade portion 101 or second blade portion 103 when implant 100 is inserted into a toe bone during a surgical procedure. In some embodiments, first blade portion 101 and second blade portion 103 are of identical shape and size. One skilled in the art will understand that first blade portion 101 and second blade portion 103 may have a variety of shapes and sizes. The shape and design of implant 100 including the shape of first blade portion 101 and second blade portion 103 prohibits joint contracture at the affected joint with implant 100 installed.

First blade portion 101 and second blade portion 103 may have a width that is greater than their thickness. Both the width and the thickness of first blade portion 101 and second blade portion 103 taper to a point 113. First blade portion 101 and second blade portion 103 may have a substantially rectangular cross-sectional area as illustrated in FIG. 1B, although one skilled in the art will understand that blade portion 103 may have other cross-sectional geometries.

Engagement portion 105 connects first blade portion 101 and second blade portion 103. Engagement portion 105 is formed from a semi-flexible material and shaped as an inverted “V” as best seen in FIG. 1B. The shape and material of engagement portion 105 allow flexibility in a joint with implant 100 installed. This flexibility allows for a more natural motion and curvature of the affected toe and alleviates many of the problems described above.

In some embodiments, engagement portion 105 is formed from silicone or a silicone composite material. In some embodiments, engagement portion 105 allows greater flexibility or range of motion in a dorsiflexion motion than in a plantar flexion motion. One skilled in the art will understand that changes to the material used to form engagement portion 105 or changes to the shape of engagement portion 105 may render implant 100 more or less flexible, and that a more or less flexible implant 100 may be desired based on the circumstances of the desired treatment for hammer toe.

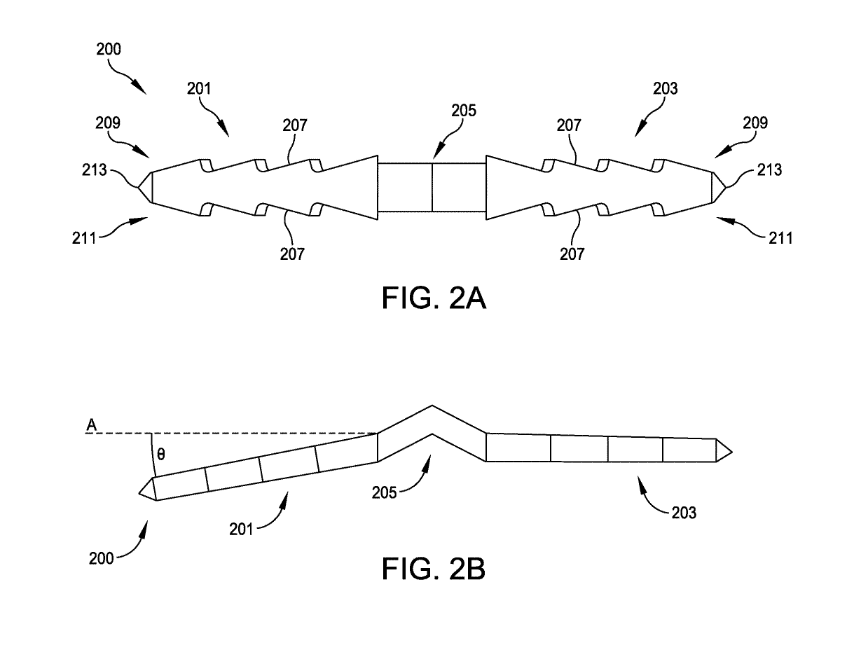

FIG. 2A is a top profile view of one example of an improved implant 200 for treating hammer toe in accordance with some embodiments of the present disclosure. FIG. 2B is a side profile view of one example of an improved implant 200 for treating hammer toe in accordance with some embodiments of the present disclosure. Similar to the example provided in FIGS. 1A and 1B, the embodiment presented in FIGS. 2A and 2B comprises a first blade portion 201 and second blade portion 203, which are connected together by an engagement portion 205. Both first blade portion 201 and second blade portion 203 have a top edge 209 and bottom edge 211 which include a plurality of serrated edges 207.

First blade portion 201 and second blade portion 203 are narrower than first blade portion 101 and second blade portion 103, which may be preferred for some applications of the implant 200. In some embodiments, as illustrated in FIG. 2B, first blade portion 201 may be disposed at angle, designated θ, with respect to a longitudinal axis A defined by second blade portion 203. The angle θ may be between zero and 45 degrees, and more particularly between approximately five and fifteen degrees, although one skilled in the art will understand that implant 200 may have other dimensions and angles.

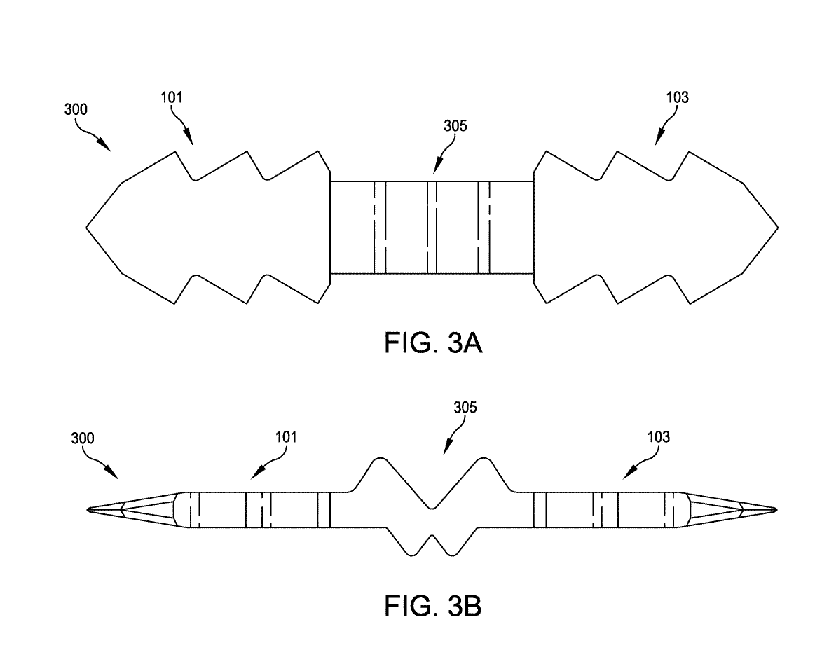

FIG. 3A is a top profile view of one example of an improved implant 300 for treating hammer toe in accordance with some embodiments of the present disclosure. FIG. 3B is a side profile view of one example of an improved implant 300 for treating hammer toe in accordance with some embodiments of the present disclosure. First blade portion 101 and second blade portion 103 are identical to those described above with regard to FIG. 1. However, in the example illustrated in FIGS. 3A and 3B, first blade portion 101 and second blade portion 103 are joined by modified engagement portion 305.

As best seen in FIG. 3B, modified engagement portion 305 is shaped substantially in a “W” shape with a pair of protrusions on either side. This shape is designed to provide limited flexibility to improved implant 300. In some embodiments, the protrusions which form the substantially “W” shape of modified engagement portion 305 prevent over-insertion of first blade portion 101 and second blade portion 103 into the affected bone.

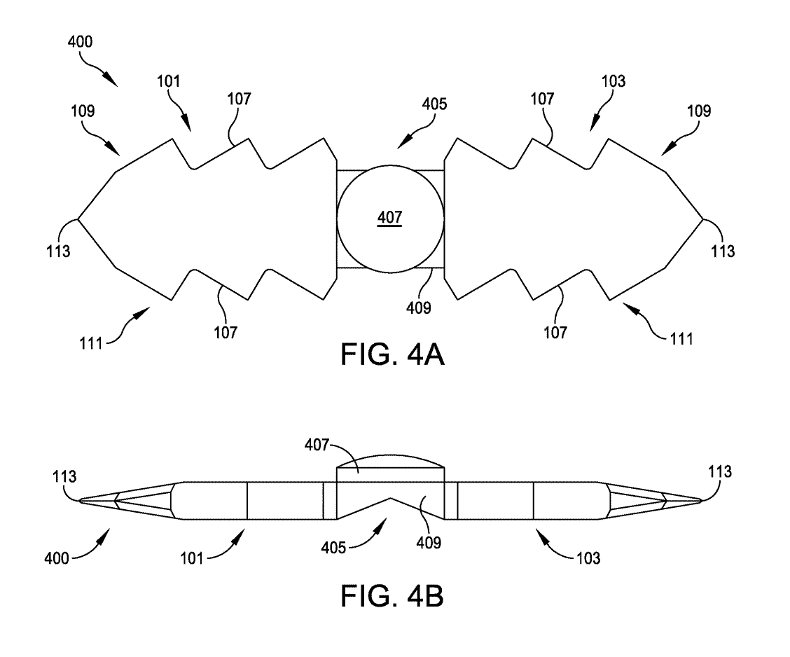

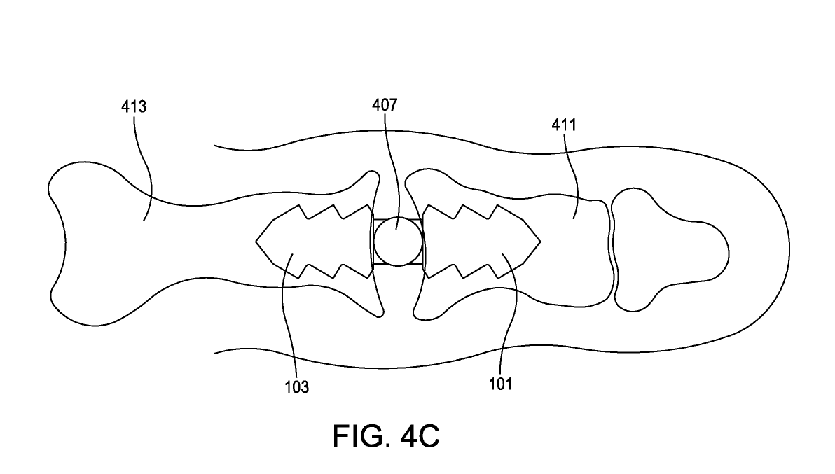

In yet another embodiment of the present disclosure, an implant may include a modified engagement portion 405 comprising a top spacer portion 407 and bottom support portion 409. FIGS. 4A and 4B illustrate such an embodiment, designated a fourth implant 400.

FIG. 4A is a top profile view of one example of an improved implant 400 for treating hammer toe in accordance with some embodiments of the present disclosure. FIG. 4B is a side profile view of one example of an improved implant 400 for treating hammer toe in accordance with some embodiments of the present disclosure. Implant 400 comprises a first blade portion 101 and second blade portion 103, which are identical to those described above with regard to FIG. 1. Implant 400 further comprises a modified engagement portion 405.

During some surgical procedures to correct or treat hammer toe, it is desired to shorten the afflicted toe bones by resection. Implant 400 includes top spacer portion 407 of engagement portion 405, which is configured to be disposed between two resected toe bones.

FIG. 4C illustrates implant 400 following a surgical procedure as it is disposed between two resected toe bones. During the surgical procedure, each of the afflicted toe bones is resected to a desired or predetermined length. First blade portion 101 is inserted into a first resected toe bone 411 and second blade portion 103 is inserted into a second resected toe bone 413. Implant 400 is positioned such that top spacer portion 407 is disposed between the resected toe bones 411 and 413, which allows the toe to move in a more natural anatomical manner than present corrective devices for hammer toe.

The implant described above may advantageously be installed through a small incision as described above. Additionally, the improved implant is completely disposed within a toe of a patient, which prevents the implant from being caught on bed sheets or other objects like the conventional pins.

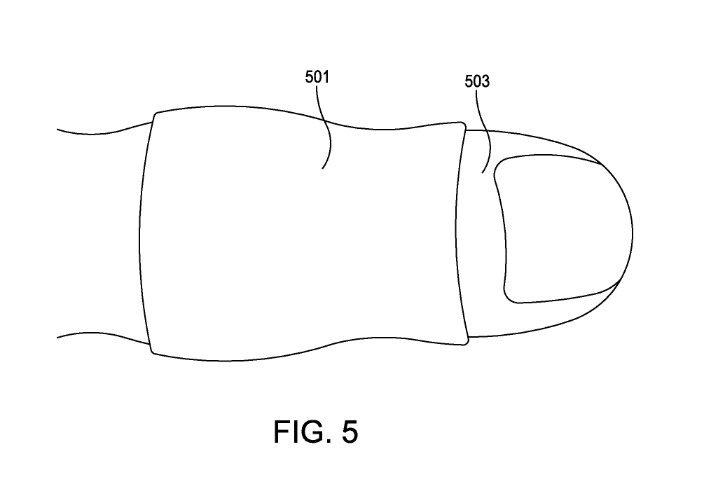

FIG. 5 is a top profile view of a sleeve 501 disposed about a circumference of the affected toe 503 in accordance with some embodiments of the present disclosure. In various embodiments, laxity of tissue about the affected joint can be an issue for various patients. In various embodiments, the sleeve 501 can be disposed to stabilize the affected joint for a predetermined period of time to improve tissue laxity. Improved tissue laxity can include a stretching of the ligaments of affected toe 503 to produce an increased laxity. In some embodiments, the predetermined period of time is an initial healing period. For example, an initial healing period could be approximately six weeks (e.g. 5-7 weeks). In some embodiments the predetermined period of time can be a period between approximately 8-12 weeks (e.g. 7-13 weeks). However, any suitable predetermined period of time can be used.

In various embodiments, a first sleeve that improves tissue laxity can be used for a first predetermined period of time and a second sleeve that is less restrictive on motion of the joint can be used for a second period of time. In some embodiments, a plurality of sleeves having descending levels of restriction of motion of the joint (e.g. from prohibiting movement to limited restriction on movement), or ascending levels of improving tissue laxity, can be used in order to provide a gradual decrease in restriction of movement and a gradual increase in permitted movement as healing of the toe progresses.

In some embodiments, sleeve 501 is disposed about a circumference of the affected toe 503 to stabilize the affected joint following surgery. In some embodiments, surgery includes the installation of implant 100 (200, 300, or 400). In still further embodiments, sleeve 501 is disposed about a circumference of the affected toe 503 to stabilize the affected joint as a conservative treatment prior to or in place of surgery.

In some embodiments, the sleeve 501 can include an aesthetically pleasing design or decoration to improve the appeal of the sleeve 501. In some embodiments, the sleeve 501 can be formed to appear as a decorative toe ring. In various embodiments, a splint (not shown) is disposed with the sleeve 501 about the affected joint to prohibit or limit motion of the joint. In various embodiments, a sleeve 501 can include a first portion that is more rigid than a second portion. In various embodiments, a sleeve 501 can include a first rigid portion to minimize media/lateral flexion and a second flexible portion to permit dorsiflexion. In some embodiments, a sleeve 501 can include a first rigid portion that is reinforced to minimize media/lateral flexion. In some embodiments, the sleeve 501 can be disposed about a circumference of the affected toe 503 prior to performance of a surgical procedure to install implant 100 (200, 300, or 400). In various embodiments, the sleeve 501 can be disposed to stretch out soft tissue in the affected toe 503 prior to installing implant 100 (200, 300, or 400) and for a predetermined period of time.

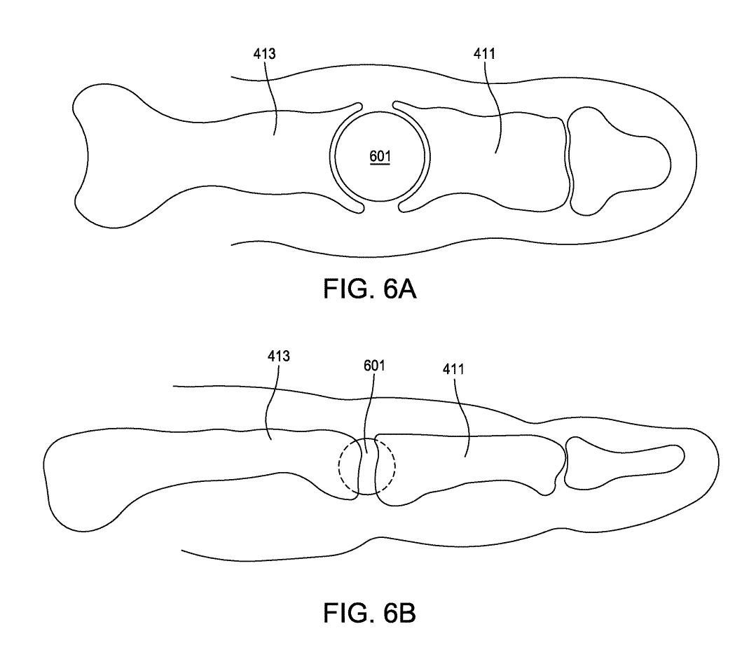

In still further aspects of the present disclosure, a spherical implant 601 is disclosed for the treatment of hammer toe. FIG. 6A is a top profile view of a spherical implant 601 disposed between two resected toe bones 411, 413 in accordance with some embodiments of the present disclosure. FIG. 6B is a side profile view of a spherical implant 601 disposed between two resected toe bones 411, 413 in accordance with some embodiments of the present disclosure.

Installing spherical implant 601 requires resection of two toe bones 411, 413 as shown in FIGS. 6A and 6B. Spherical implant 601 is then installed between the resected bones to act as a spacer. Resected bones 411 and 413, with spherical implant 601 disposed between them, are permitted to move in a natural anatomical manner following post-surgery healing.

In some embodiments, spherical implant 601 is formed from a silicone or a silicone composite material. In other embodiments, spherical implant is formed from stainless steel or similar material.

In some embodiments, spherical implant 601 is not connected to resected bones 411, 413 with any adhesive, sutures, or similar material. Following post-surgery healing, spherical implant 601 is held between resected bones 411 and 413 by the surrounding healed tissue including without limitation the surrounding ligaments.

In some embodiments, an implant comprises a first blade portion comprising a plurality of serrated edges; a second blade portion comprising a plurality of serrated edges; and an engagement portion connecting the first blade portion and the second blade portion, wherein the first blade portion and the second blade portion are aligned along a common axis extending away from the engagement portion and both the first blade portion and the second blade portion have a taper terminating at a point. In some embodiments, at least one of the first blade portion and the second blade portion tapers along its width and thickness to the point. In some embodiments, the second blade portion is of substantially identical size and shape as the first blade portion. In some embodiments, the engagement portion is formed from a semi-flexible material. In some embodiments, the engagement portion is formed in an inverted ‘V’ shape. In some embodiments, the engagement portion is formed in an inverted ‘W’ shape. In some embodiments, the engagement portion comprises a top spacer portion configured to be disposed between two resected toe bones. In some embodiments, the engagement portion is formed from a silicone-based material.

In some embodiments, an implant comprises a first blade portion; a second blade portion; and an engagement portion connecting the first blade portion and the second blade portion, wherein the first blade portion extends from the engagement portion at an angle with respect to an axis defined by the second blade portion and both the first blade portion and the second blade portion have a taper terminating at a point. In some embodiments, the angle is between zero and 45 degrees. In some embodiments, at least one of the first blade portion and the second blade portion includes a plurality of serrated edges. In some embodiments, the engagement portion comprises a top spacer portion configured to be disposed between two resected toe bones. In some embodiments, the engagement portion is formed from a semi-flexible material and in an inverted ‘V’ shape. In some embodiments, the engagement portion is formed from a semi-flexible material and in an inverted ‘W’ shape. In some embodiments, the engagement portion is formed from a silicone-based material. In some embodiments, the engagement portion is formed from a silicone-based material.

In some embodiments, a method comprises exposing a joint between first and second bones; resecting a respective end of the first and second bones; and installing an implant in the joint, the implant comprising: a first blade portion having a first taper terminating at a first point and a plurality of serrated edges configured to engage the first bone; a second blade portion having a second taper terminating at a second point and a plurality of serrated edges configured to engage the second bone; and an engagement portion connecting the first blade portion and the second blade portion, the engagement portion having a top spacer portion configured to be disposed between the first and second bones when the implant is installed. In some embodiments, the method further comprises disposing a sleeve about the joint wherein the sleeve is configured to limit motion in a first predetermined direction for a predetermined period of time. In some embodiments, the sleeve comprises a first portion configured to limit rotation in a medial/lateral direction and a second portion configured to permit dorsiflexion motion. In some embodiments, the first blade portion extends from the engagement portion at an angle with respect to an axis defined by the second blade portion.

In some embodiments, a method, comprises exposing a joint between first and second bones; resecting a respective end of the first and second bones; installing a spherical implant in the joint; and disposing a sleeve about the joint wherein the sleeve is configured to limit motion in a first predetermined direction for a predetermined period of time and wherein the spherical implant is configured to be held in the joint by surrounding tissue subsequent to expiration of the predetermined period of time. In some embodiments, the sleeve comprises a first portion configured to limit rotation in a medial/lateral direction and a second portion configured to permit dorsiflexion motion.

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

What is claimed is:

1. An implant, comprising:
a first blade portion comprising a plurality of serrated edges;
a second blade portion comprising a plurality of serrated edges; and
an engagement portion connecting the first blade portion and the second blade portion, wherein the first blade portion and the second blade portion are aligned along a common axis extending away from the engagement portion and both the first blade portion and the second blade portion have a taper terminating at a point.

2. The implant of claim 1, wherein at least one of the first blade portion and the second blade portion tapers along its width and thickness to the point.

3. The implant of claim 2, wherein the second blade portion is of substantially identical size and shape as the first blade portion.

4. The implant of claim 3, wherein the engagement portion is formed from a semi-flexible material.

5. The implant of claim 4, wherein the engagement portion is formed in an inverted ‘V’ shape.

6. The implant of claim 4, wherein the engagement portion is formed in an inverted ‘W’ shape.

7. The implant of claim 4, wherein the engagement portion comprises a top spacer portion configured to be disposed between two resected toe bones.

8. The implant of claim 4, wherein the engagement portion is formed from a silicone-based material.

9. An implant, comprising:
a first blade portion;
a second blade portion; and
an engagement portion connecting the first blade portion and the second blade portion, wherein the first blade portion extends from the engagement portion at an angle with respect to an axis defined by the second blade portion and both the first blade portion and the second blade portion have a taper terminating at a point.

10. The implant of claim 9, wherein the angle is between zero and 45 degrees.

11. The implant of claim 10, wherein at least one of the first blade portion and the second blade portion includes a plurality of serrated edges.

12. The implant of claim 11, the engagement portion comprises a top spacer portion configured to be disposed between two resected toe bones.

13. The implant of claim 10, wherein the engagement portion is formed from a semi-flexible material and in an inverted ‘V’ shape.

14. The implant of claim 10, wherein the engagement portion is formed from a semi-flexible material and in an inverted ‘W’ shape.

15. The implant of claim 13, wherein the engagement portion is formed from a silicone-based material.

16. The implant of claim 14, wherein the engagement portion is formed from a silicone-based material.

17. A method, comprising:
exposing a joint between first and second bones;
resecting a respective end of the first and second bones; and
installing an implant in the joint, the implant comprising:
a first blade portion having a first taper terminating at a first point and a plurality of serrated edges configured to engage the first bone;
a second blade portion having a second taper terminating at a second point and a plurality of serrated edges configured to engage the second bone; and
an engagement portion connecting the first blade portion and the second blade portion, the engagement portion having a top spacer portion configured to be disposed between the first and second bones when the implant is installed.

18. The method of claim 17, further comprising:
disposing a sleeve about the joint wherein the sleeve is configured to limit motion in a first predetermined direction for a predetermined period of time.

19. The method of claim 18, wherein the sleeve comprises a first portion configured to limit rotation in a medial/lateral direction and a second portion configured to permit dorsiflexion motion.

20. The method of claim 17, wherein the first blade portion extends from the engagement portion at an angle with respect to an axis defined by the second blade portion.

21. A method, comprising:
exposing a joint between first and second bones;
resecting a respective end of the first and second bones;
installing a spherical implant in the joint; and
disposing a sleeve about the joint wherein the sleeve is configured to limit motion in a first predetermined direction for a predetermined period of time and wherein the spherical implant is configured to be held in the joint by surrounding tissue subsequent to expiration of the predetermined period of time.

22. The method of claim 21, wherein the sleeve comprises a first portion configured to limit rotation in a medial/lateral direction and a second portion configured to permit dorsiflexion motion.
